(19) United States
(12) Patent Application Publication
(10) Pub. No.: US 2020/0069618 A1
(43) Pub. Date: Mar. 5, 2020

(54) COMPOSITIONS HAVING AN AGENT AND AN ENHANCER THEREOF, METHODS OF USE, AND DELIVERY SYSTEMS

(71) Applicants: John C. Gustin, Carlsbad, CA (US); Michael J. McCarthy, Oceanside, CA (US)

(72) Inventors: John C. Gustin, Carlsbad, CA (US); Michael J. McCarthy, Oceanside, CA (US)

(21) Appl. No.: 16/559,614

(22) Filed: Sep. 3, 2019

Related U.S. Application Data
(60) Provisional application No. 62/726,961, filed on Sep. 4, 2018.

(51) Int. Cl. A61K 31/164; A61K 31/232; A61K 31/352; A61K 31/015

(52) U.S. Cl. CPC A61K 31/164; A61K 31/015; A61K 31/352; A61K 31/232

(57) ABSTRACT

The present invention relates to compositions, and methods of use thereof, related to the endocannabinoid system and includes therapeutic compositions including an agent and an enhancer thereof, optionally, formulated for administration of the therapeutic compositions, preferably in a measured amount.

Claims Priority to: U.S. Provisional Application 62/726,961 Filed Sep. 4, 2018

### BACKGROUND OF THE INVENTION

The present invention is in the technical field of biochemistry. More particularly, the present invention is in the technical field of cannabinoid and endocannabinoid biochemistry.

### SUMMARY OF THE INVENTION

The present invention provides compositions, articles of manufacture and methods of use thereof, comprising at least one cannabinoid receptor agent and at least one enhancer of an activity of the cannabinoid receptor agent.

### BRIEF DESCRIPTION OF THE INVENTION

One embodiment of the present invention provides a therapeutic composition, comprising: a cannabinoid receptor agent and a cannabinoid enhancer. In certain embodiments, the composition further comprises a carrier suitable for delivery of the composition to a mammal, preferably a human.

In certain embodiments, a cannabinoid receptor agent is anandamide, 2-arachidonylglycerol or caryophyllene. In certain embodiments, a cannabinoid enhancer inhibits fatty acid amide hydrolase or monoacylglycerol lipase, is a cannabinoid reuptake inhibitor (CRI). In certain embodiments, the cannabinoid receptor agent is an anandamide and the cannabinoid enhancer is a cannabinol or a cannabidiol. In certain embodiments, the cannabinoid receptor agent is a 2-arachidonylglycerol and the cannabinoid enhancer is a cannabinol or a cannabidiol. In certain embodiments, the cannabinoid receptor agent is a caryophyllene and the cannabinoid enhancer is a cannabinol or a cannabidiol.

One embodiment of the present invention provides an article of manufacture, comprising: a device adapted for administration of a metered or measured dose of therapeutic composition to a subject in need of therapy, wherein the therapeutic is a composition of the present invention. In certain embodiments, the device is a metered dose device. Metered dosing is known in the art. In certain embodiments, the device is a medical device. In certain embodiments, the cannabinoid receptor agent is anandamide, 2-arachidonylglycerol or caryophyllene and the cannabinoid enhancer inhibits fatty acid amide hydrolase or monoacylglycerol lipase. In certain embodiments, the cannabinoid receptor agent is an anandamide and the cannabinoid enhancer is a cannabinol or a cannabidiol. In certain embodiments, the cannabinoid receptor agent is a 2-arachidonylglycerol and the cannabinoid enhancer is a cannabinol or a cannabidiol.

In certain embodiments, a composition of the present invention includes at least 5 mg of a cannabinoid receptor agent and at least 3 mg of an enhancer thereof. In certain embodiments, a composition of the present invention includes at least 10 mg of a cannabinoid receptor agent and at least 10 mg of an enhancer thereof. In certain embodiments, a composition of the present invention includes at least 20 mg of a cannabinoid receptor agent and at least 5 mg of an enhancer thereof.

Synergism

In certain embodiments, it is an aspect of the invention that a composition including both a cannabinoid receptor agent and a cannabinoid enhancer yields a synergistic effect when administered as a therapeutic remedy. A synergist effect occurs because an action of the receptor agent activates a binding or a receptor signal (e.g., a biochemical signal) and an effect of the enhancer maintains the signal over a longer period of time or increases the amount or amplitude of the signal. In certain embodiments, the enhancer inhibits the degradation of the cannabinoid receptor agent. In certain embodiments, the enhancer inhibits the degradation of the cannabinoid receptor agent by inhibiting an enzyme that breaks down the agent or protects the agent, such as by physical interaction or chemical protection.

In one embodiment, an anandamide (AEA) activates a cannabinoid receptor (e.g., a CB1 receptor), to produce a biochemical signal and the cannabinoid enhancer, (e.g., cannabidiol), blocks the breakdown of the AEA (the agent) by inhibiting, for example, a fatty acid amide hydrolase (FAAH) enzyme which has a biochemical action of metabolizing the breakdown of the agent (e.g., AEA). This combination of agent and enhancer yields a synergy of action because the combination composition causes the AEA, for example, to continue to activate the receptor for a longer period of time, causes an increased local concentration of AEA, for example which binds more receptors because the local agent concentration at the receptor continues to increase for a period of time.

Medicaments

Certain embodiments of the present invention provide a medicament for enhancing a wellbeing, treating a disease, treating a condition or treating a disorder in a mammal, wherein the medicament comprises: a cannabinoid agent and a cannabinoid enhancer, in combination. In certain embodiments, the medicament is a capsule containing the combination. In certain embodiments, the medicament is a tablet formed with the combination. In a preferred embodiment the mammal is a human.

In certain embodiments, the medicament is a liquid containing the combination (for example, optionally, as an injectable or an oral liquid). In certain embodiments, the medicament is a lyophilized powder containing the combination. In certain embodiments, the medicament optionally includes an excipient, a forming agent, a filler, a binder or the like; as may be useful for production of the medicament. Many suitable excipients are known in the art.

In certain embodiments, the medicament is an aqueous or hydrophilic formulation. In certain embodiments, the medicament is a hydrophobic formulation. In certain embodiments, the medicament is an amphiphilic formulation (possessing both hydrophilic (water-loving, polar) and lipophilic (fat-loving, non-polar) properties). The formulation produced will depend on the physical chemical characteristics of the agent(s) or enhancer(s) selected for a particular medicament as known in the art.

In certain embodiments, the medicament further comprises in formulation one or more of: a nanoparticle, a biopolymer, a pharmaceutically acceptable propellant, a micelle (i.e., one or more lipids combined to form a micelle), a liposome (i.e., one or more lipids combined to form a liposome) or a combination thereof.

Dosage

In certain embodiments, the dose of the cannabinoid agent or the cannabinoid enhancer (or both) is optimized to provide enhanced wellbeing or for treatment of a disease, disorder or condition in a subject (mammal, preferably a human). In certain preferred embodiments, a subject (themselves) or a healer, health practitioner, or physician caring for or treating the subject, selects a predetermined amount, measurement or dose of a composition of the present invention for administration to the subject. In certain embodiments, a device is provided that can administer user selectable or predetermined doses of a composition of the present invention.

Certain Embodiments of a Dosage Regime

In certain embodiments, a cannabinoid agent or an enhancer of the present invention is provided in a dose selected from the following:

In certain embodiments, a cannabinoid agent is provided in a dose of 1 mg or more.

In certain embodiments, a cannabinoid agent is provided in a dose of 2 mg or more.

In certain embodiments, a cannabinoid agent is provided in a dose of 3 mg or more.

In certain embodiments, a cannabinoid agent is provided in a dose of 5 mg or more.

In certain embodiments, a cannabinoid agent is provided in a dose of 7 mg or more.

In certain embodiments, a cannabinoid agent is provided in a dose of 10 mg or more.

In certain embodiments, a cannabinoid agent is provided in a dose of 15 mg or more.

In certain embodiments, a cannabinoid agent is provided in a dose of 20 mg or more.

In certain embodiments, a cannabinoid agent is provided in a dose of 25 mg or more.

In certain embodiments, a cannabinoid agent is provided in a dose of 30 mg or more.

In certain embodiments, a cannabinoid agent is provided in a dose of 40 mg or more.

In certain embodiments, a cannabinoid agent is provided in a dose of 50 mg or more.

In certain embodiments, a cannabinoid agent is provided in a dose of 75 mg or more.

In certain embodiments, a cannabinoid agent is provided in a dose of 100 mg or more.

In certain embodiments, a cannabinoid agent is provided in a dose of 150 mg or more.

In certain embodiments, a cannabinoid agent is provided in a dose of 200 mg or more.

In certain embodiments, a cannabinoid agent is provided in a dose of 250 mg or more.

In certain embodiments, a cannabinoid agent is provided in a dose of 300 mg or more.

In certain embodiments, a cannabinoid agent is provided in a dose of 400 mg or more.

In certain embodiments, a cannabinoid agent is provided in a dose of 500 mg or more.

In certain embodiments, an enhancer is provided in a dose of 1 mg or more.

In certain embodiments, an enhancer is provided in a dose of 2 mg or more.

In certain embodiments, an enhancer is provided in a dose of 3 mg or more.

In certain embodiments, an enhancer is provided in a dose of 5 mg or more.

In certain embodiments, an enhancer is provided in a dose of 7 mg or more.

In certain embodiments, an enhancer is provided in a dose of 10 mg or more.

In certain embodiments, an enhancer is provided in a dose of 15 mg or more.

In certain embodiments, an enhancer is provided in a dose of 20 mg or more.

In certain embodiments, an enhancer is provided in a dose of 25 mg or more.

In certain embodiments, an enhancer is provided in a dose of 30 mg or more.

In certain embodiments, an enhancer is provided in a dose of 40 mg or more.

In certain embodiments, an enhancer is provided in a dose of 50 mg or more.

In certain embodiments, an enhancer is provided in a dose of 75 mg or more.

In certain embodiments, an enhancer is provided in a dose of 100 mg or more.

In certain embodiments, an enhancer is provided in a dose of 150 mg or more.

In certain embodiments, an enhancer is provided in a dose of 200 mg or more.

In certain embodiments, an enhancer is provided in a dose of 250 mg or more.

In certain embodiments, an enhancer is provided in a dose of 300 mg or more.

In certain embodiments, an enhancer is provided in a dose of 400 mg or more.

In certain embodiments, an enhancer is provided in a dose of 500 mg or more.

In certain embodiments, it is desirable to provide a low dose of an agent and enhancer combination, wherein the minimal dosage of the enhancer and the agent is 1 mg, 2, mg, 3 mg, 4 mg, 5 mg, 6 mg, 7 mg, 8 mg, 9 mg, or 10 mg (each) and the maximal dose of each, the agent and the enhancer, is set forth in the section labeled “Certain Embodiments of a Dosage Regime,” excepting the 1 mg example in the Dosage Regime. A low dose schema is desirable in certain embodiments where the agent/enhancer has a beneficial effect (for example, on one or more of: a cognitive improvement, a neuroprotection, a relief of pain, a reduction in an inflammation or an inflammatory response, a neuro-proliferation, an improvement in neuron health, an improvement of a symptom of a neurodegenerative condition, a reduction in a symptom of a mental illness including depression, schizophrenia or psychosis, a reduction in anxiety, an increase in sleep duration or quality or both, a reduction in a symptom of insomnia, a reduction in a symptom of a headache, a reduction in a symptom of a migraine, or a weight gain in a subject in need of or desirous of a weight gain); but, the administration of a larger dose or amount of either the agent or the enhancer, or both leads to an increase in an undesirable symptom, effect or result (for example, a weight gain in a subject that is not in need or desirous of a weight gain, a high or an intoxication, a decrease in mental acuity or cognitive performance, a decrease in a memory performance or recall, or other effects and the like associated with the use of Cannabis, in general).

Cannabinoid

The meaning of cannabinoid, herein, is meant to include, in certain embodiments, phytocannabinoids and endocannabinoids. The meaning of phytocannabinoid, herein, is meant to refer to cannabinoid molecules from plant sources, such as hemp or cannabis. The meaning of endocannabinoid, herein, is meant to refer to compounds of the mammalian endocannabinoid system (for example, anandamide and 2-arachidonylglycerol). In certain embodiments, cannabinoid refers to phytocannabinoids alone and in certain other embodiments, cannabinoid refers to endocannabinoids alone. In certain embodiments, caryophyllene understood to be a useful optional factor for selected formulations and is optionally designated as a cannabinoid for purposes of certain embodiments of the present invention.

Examples of Cannabinoid Agents

In certain embodiments, the following are exemplary cannabinoid agents of the present invention and can include, but are not limited to:

Anandamide (AEA)

2-arachidonylglycerol (2-AG)

Caryophyllene

2-Oleoylglycerol (2-OG)

Cannabidiol

Cannabinol

URB597

OL-135

ST4070

NJ-1661010

OL-92

Fluorophosphonates, such as, MAFP and IDFP

AM6701

N,N-dimethyl-5-(4-phenoxyphenyl)-2H-tetrazole-2-carboxamide

JZL184

MJN110

KML29

JW651

AM6701

N,N-dimethyl-5-(4-phenoxyphenyl)-2H-tetrazole-2-carboxamide

N-((1-(1H-1,2,4-triazole-1-carbonyl)piperidin-4-yl) methyl)-4-chlorobenzenesulfonamide

1,1,1,3,3,3-hexafluoropropan-2-yl 4-(((4-chlorophenyl)sulfonamido) methyl)piperidine-1-carboxylate

AM404

AM1172

LY-2183240

O-2093

OMDM-2

UCM-707

VDM-11

URB597

An agonist of CB1, CB2, TRPV1 (vanilloid receptor), N-arachidonoyl glycine (NAGly) receptor, GPR18, GPR55 or GPR119

A tetrahydrocannabinol (THC)

A carmabigerolic acid (CBGA)

A cannabigerol (CBG)

A tetrahydrocannabinolic acid (THCA)

A cannabichromene (CBC)

A cannabicyclol (CBL)

A cannabivarin (CBV)

A cannabichromevarin (CBCV)

A cannabigerovarin (CBGV)

A cannabigerol Monomethyl Ether (CBGM)

A delta-8-tetrahydrocannabinol (D8THC)

A delta-9-tetrahydrocannabinol (D9THC)

A tetrahydrocannabivarin (THCV)

A cannabinolic acid (CBNA)

A cannabinol (CBN)

A cannabidiolic acid (CBDA)

A cannabidivaric acid (CBDVA)

A cannabidiol (CBD)

A cannabichromenic acid (CBCA)

A cannabichromene (C BC)

A cannabicyclolic acid (CBLA)

A stereo isomer of a cannabinoid

A salt of a cannabinoid agent of the present invention.

Examples of Enhancers

In certain embodiments, the following are exemplary enhancers of the present invention which can include, but are not limited to:

A cannabinoid agent reuptake inhibitor (CARI)

A fatty acid amide hydrolase (FAAH) inhibitor

A monoacylglycerol lipase (MAGL) inhibitors

An endocannabinoid transporter (eCBT) inhibitor

An endocannabinoid reuptake inhibitor (eCBRI)

Dihomo-g-linolenoyl ethanolamide

docosatetraenoyl ethanolamide

Cannabidiol (CBD)

2-arachidonyl glycerol ether

0-arachidonoylethanolamide

n-arachidonoyldopamine

N-acylethanolamines

palmitoylethanolamide (PEA)

oleoylethanolamide (OEA)

N-linoleoylethanolamine (18:3 NAE)

A salt of an enhancer of the present invention.

Certain Embodiments of Agent and Enhancer Combinations

In certain preferred embodiments, after an agent is selected for a formulation having one or more effects on an indication (e.g., a disease or condition), then an enhancer is selected which increases the effect(s) of the agent when administered in combination. For example, the enhancer can reduce the breakdown of the agent, increase the affinity of the agent and the receptor of the agent (in the case that the agent acts through a receptor), and the like. In certain embodiments, a cannabidiol compound can act as a cannabinoid agent or as an enhancer of a cannabinoid agent. In certain embodiments, a cannabidiol compound cannot be both the agent and the enhancer in the same composition, it is used as either the cannabinoid agent or the enhancer of another cannabinoid agent.

Certain Embodiments Having Exclusions

In certain embodiments, it is desirable, for legal reasons, that the cannabinoid agent and the cannabinoid enhancer does not include a compound classified by a US regulating authority as a tetrahydrocannabinol. In certain embodiments, it is desirable, for legal reasons, that the cannabinoid agent and the cannabinoid enhancer does not include a compound classified by a US regulating authority as a cannabis-derived compound. In certain embodiments, it is desirable, for legal reasons, that the cannabinoid agent and the cannabinoid enhancer does not include a compound classified by a US regulating authority as a hemp-derived compound. As can be understood by the description of the present invention, not all compounds useful for the present invention are a tetrahydrocannabinol, cannabis-derived or hemp-derived.

Subjects—Patients

In certain embodiments, a subject or patient can be a mammal, preferably a person in need of treatment for enhancing a wellbeing or for improving a disease, disorder or condition.

In certain embodiments, it is desired to enhance a bliss or a sense of wellbeing in a subject as a need for therapy. For example, a lack of wellbeing may be a symptom of depression or other disorders, particularly mental disorders. Mental disorders include: depression, bi-polar disorder, schizophrenia, affective disorder, personality disorder and post-traumatic stress disorder (PTSD).

Composition Components Binding, Activating or Agonist

In certain embodiments, a cannabinoid receptor agent binds to a cannabinoid receptor. In certain embodiments, a cannabinoid receptor agent is an agonist of a cannabinoid receptor. In preferred embodiments, a cannabinoid receptor agent binds to and activates a cannabinoid receptor. In certain embodiments, a receptor is a molecule or complex of molecules which receives one or more upstream biological signals and transduces the one or more upstream biological signals into one or more downstream biological signals.

In certain embodiments, receptors can be stimulated by either endogenous (such as hormones and neurotransmitters) or exogenous (such as drugs which originate from outside the body) signals or ligands and result in a biological response. In certain embodiments, examples of the types of upstream receptors signals include:
Agonists,
Inducers,
Activators,
Superagonists,
Full Agonists,
Partial Agonists,
Inverse Agonists,
Co-Agonists,
Irreversible Agonists,
Selective Agonists,
Antagonists,
Inhibitors,
Blockers,
Competitive Antagonists,
Non-competitive Antagonists,
Uncompetitive Antagonists,
Partial Antagonists, and
Inverse Antagonists.

Certain Embodiments Having Selected Routes of Administration

In preferred embodiments, the therapeutic composition is administered to a subject in need of such administration for treatment of a condition or a symptom of a condition. The terms illness, disease and condition can be used interchangeably in certain embodiments herein.

In certain embodiments, examples of routes of administration of a composition of the present invention include administration by: intranasal, suppository, rectal insertion, transdermal absorption, intraperitoneal injection or intraperitoneal pump infusion, subcutaneous injection or subcutaneous pump infusion, oral delivery (the composition can be administered orally as, for example, as a: liquid, capsule, tablet or chewable tablet), injection, sublingually, buccal, rectal, vaginal, ocular, or otic (into the ear). In certain embodiments, a composition of the present invention can be administered by any method known in the art. In certain embodiments, a composition of the present invention can further include an expedient, preservative, absorption factor, etc. as are known in the art. Certain embodiments of the present invention are described below, but do not limit the choice of a method of delivery that is known in the art.

Intranasal Route

One embodiment of the present invention provides an article of manufacture, comprising: a device adapted for administration of a therapeutic composition to a nasal cavity, wherein the therapeutic composition includes a cannabinoid receptor agent and a cannabinoid enhancer. In certain embodiments, the device is a metered dose device. In certain embodiments, the device is a medical device. In certain embodiments, the cannabinoid receptor agent is anandamide, 2-arachidonylglycerol or caryophyllene and the cannabinoid enhancer inhibits fatty acid amide hydrolase or monoacylglycerol lipase. In certain embodiments, the cannabinoid receptor agent is an anandamide and the cannabinoid enhancer is a cannabinol or a cannabidiol. In certain embodiments, the cannabinoid receptor agent is a 2-arachidonylglycerol and the cannabinoid enhancer is a cannabinol or a cannabidiol.

In certain embodiments, a medicament comprises is a liquid containing the combination (for example, optionally, as an injectable, a nasal or an oral liquid). In certain embodiments, the medicament is a lyophilized powder containing the combination. In certain embodiments, the medicament is prepared for intranasal administration. In certain embodiments, the medicament optionally includes an excipient, a forming agent, a filler, a binder or the like; as may be useful for production of the medicament. Suitable excipients for intranasal administration optionally include an hydrofluoroalkane or an aqueous preparation.

In preferred embodiments, the therapeutic composition is administered to the nasal cavity or a mammal, preferably a livestock or pet, and more preferably, a human. The terms nasal administration, nasal delivery, intranasal administration and intranasal delivery can be used interchangeably in certain embodiments herein.

Nasal Route

In certain embodiments, a composition of the present invention is delivered by administration to the mucous membrane that lines the nasal passages (i.e., intranasal administration). In certain embodiments, the composition is transformed into tiny droplets in air (atomized) or administered with a propellant in aqueous, oil, or in powder form. Once administered, the composition can enter the bloodstream, enter into the brain or both. Drugs administered by the nasal route generally work quickly and, in certain embodiments, can pass through the brain blood barrier for enhanced delivery to the central nervous system, including the brain (for example, by moving along or within the olfactory nerve).

Oral Route

Many drugs can be administered orally as liquids, capsules, tablets, or chewable tablets. Because the oral route is the convenient, it is often used. However, it has limitations because of the way a drug typically moves through the digestive tract. For drugs administered orally, absorption may begin in the mouth and stomach. However, most drugs are usually absorbed from the small intestine. The drug passes through the intestinal wall and travels to the liver where it might be metabolized into breakdown products before being transported via the bloodstream to its target site. The intestinal wall and liver chemically alter (metabolize) many drugs, decreasing the amount of drug reaching the bloodstream. Conversely, the metabolism can convert a pro-drug into the active agent or enhancer. Consequently, these drugs are often given in smaller doses when injected intravenously to produce the same effect.

When a drug is taken orally, food and other drugs in the digestive tract may affect how much of and how fast the drug is absorbed. Thus, some drugs should be taken on an empty stomach, others should be taken with food and others should not be taken with certain other drugs.

Some orally administered drugs irritate the digestive tract. For example, aspirin and most other nonsteroidal anti-inflammatory drugs (NSAIDs) can harm the lining of the stomach and small intestine to potentially cause or aggravate preexisting ulcers. Other drugs are absorbed poorly or erratically in the digestive tract or are destroyed by the acid and digestive enzymes in the stomach.

Other routes of administration are required when the oral route cannot be used, for example: when a person cannot take anything by mouth, when a drug must be administered rapidly or in a precise or very high dose, or when a drug is poorly or erratically absorbed from the digestive tract.

Injection Routes

Administration by injection (parenteral administration) includes the following routes: subcutaneous (under the skin), intramuscular (in a muscle), intravenous (in a vein), intrathecal (around the spinal cord). A drug product can be prepared or manufactured in ways that prolong drug absorption from the injection site for hours, days, or longer. Such products do not need to be administered as often as drug products with more rapid absorption. Such methods and materials are known in the art.

In certain embodiments, a subcutaneous route is preferred. For example, a needle is inserted into fatty tissue just beneath the skin. After a drug is injected, it then moves into small blood vessels (capillaries) and is carried away by the bloodstream. Alternatively, a drug reaches the bloodstream through the lymphatic vessels. Protein drugs that are large in size, such as insulin, usually reach the bloodstream through the lymphatic vessels because these drugs move slowly from the tissues into capillaries. The subcutaneous route is used for many protein drugs when such drugs are destroyed in the digestive tract when they are taken orally.

Certain drugs may be given by inserting plastic capsules under the skin (implantation). Although this route of administration is used infrequently in the art, its main advantage is to provide a long-term therapeutic effect (for example, etonogestrel that is implanted for contraception can last up to 3 years).

In certain embodiments, an intramuscular delivery route is preferred to the subcutaneous route when larger volumes of a drug product are needed. Because the muscles lie below the skin and fatty tissues, a longer needle is used. Drugs are usually injected into the muscle of the upper arm, thigh, or buttock. How quickly the drug is absorbed into the bloodstream depends, in part, on the blood supply to the muscle: The sparser the blood supply, the longer it takes for the drug to be absorbed.

In certain embodiments, an intravenous route is preferred. For example, a needle is inserted directly into a vein. A solution containing the drug may be given in a single dose or by continuous infusion. For infusion, the solution is moved by gravity (from a collapsible plastic bag) or, more commonly, by an infusion pump through thin flexible tubing to a tube (catheter) inserted in a vein, usually in the forearm. Intravenous administration is the best way to deliver a precise dose quickly and in a well-controlled manner throughout the body. It is also used for irritating solutions, which would cause pain and damage tissues if given by subcutaneous or intramuscular injection.

When given intravenously, a drug is delivered immediately to the bloodstream and tends to take effect more quickly than when given by any other route. Consequently, health care practitioners closely monitor people who receive an intravenous injection for signs that the drug is working or is causing undesired side effects. Also, the effect of a drug given by this route tends to last for a shorter time. Therefore, some drugs may be given by continuous infusion to keep their effect constant.

In certain embodiments, an intrathecal route is preferred. For example, a needle is inserted between two vertebrae in the lower spine and into the space around the spinal cord. The drug is then injected into the spinal canal. A small amount of local anesthetic is often used to numb the injection site. This route is used when a drug is needed to produce rapid or local effects on the brain, spinal cord, or the layers of tissue covering them (meninges), for example, to treat infections of these structures. Anesthetics and analgesics (such as morphine) are sometimes given this way.

In certain embodiments, a sublingual or a buccal route is preferred. For example, a drug may be placed under the tongue (taken sublingually) or between the gums and teeth (taken bucally) so that they can dissolve and be absorbed directly into the small blood vessels that lie beneath the tongue or in the gums and cheeks. Preferably, these drugs are not swallowed. The sublingual route is especially good for nitroglycerin, for example, which is used to relieve angina, because absorption is rapid and the drug immediately enters the bloodstream without first passing through the intestinal wall and liver.

In certain embodiments, a rectal delivery is preferred. For example, a drug can be inserted into the rectum. In one example, as a suppository. In this form, a drug is typically mixed with a waxy substance that dissolves or liquefies after it is inserted into the rectum (e.g., by contact with moisture or temperature). Because the rectum's wall is thin and its blood supply rich, the drug is readily absorbed. An enema can also be used as a route of rectal drug administration.

In certain embodiments, a composition of the present invention is administered to a woman by vaginal insertion. In certain examples, a composition may be administered vaginally to women as a solution, tablet, cream, gel, suppository, or ring. This route is often used to give estrogen to women during menopause to relieve vaginal symptoms such as dryness, soreness, and redness.

In certain embodiments, a composition of the present invention is administered via ocular delivery (e.g., absorption into the fluid in the eye, such as by eye drops). Compounds used to treat eye disorders (including glaucoma, conjunctivitis, and injuries) can be mixed with excipients to make a liquid, gel, or ointment suitable for application to the eyes. Ocular delivery is preferably used for local delivery to the eye, such as for treatment of glaucoma or reduction in a symptom of glaucoma, as used in certain embodiments herein.

In certain embodiments, a composition of the present invention is administered via an otic delivery route. For example, a composition can be applied directly to one or both ears. Ear drops containing solutions or suspensions are typically applied only to the outer ear canal. Typically, little of the a composition of the present invention enters the bloodstream as a result of otic delivery, so body-wide side effects are typically minimal or absent. Accordingly, in certain embodiments that include otic delivery systems, the effect is intended to result in local treatment in the ear, for example, for improving the function of hearing or improving the function of nerves or nerve tissue involved in hearing such as the otic nerve.

In certain embodiments, a composition of the present invention is administered via inhalation, such as by pulmonary inhalation. In certain embodiments, a composition of the present invention that is administered by inhalation through the mouth are atomized into smaller droplets or particles, so that the composition can pass through the windpipe (trachea) and into the lungs. How deeply into the lungs the droplets or particles go depends on their size. Smaller droplets and particles go deeper into the lungs than larger droplets or particles. In certain embodiments, droplets or particles smaller than 3 microns or, sometimes preferred, smaller than 2.5 microns can be delivered into the alveolar air sacs, which can increase the amount of the composition absorbed into the bloodstream through the lung tissues and vessels.

In certain embodiments, a composition of the present invention is nebulized prior to administration. In certain embodiments, a composition of the present invention, as administered by nebulization are aerosolized into small particles to reach deep into the lungs. Nebulization can make use of special devices, most commonly ultrasonic or jet nebulizer systems. Using nebulization devices can increase the amount delivered for a composition of the present invention.

In certain embodiments, a composition of the present invention is delivered via cutaneous administration.

In certain embodiments, such administration, is used to treat skin disorders, such as psoriasis, eczema, skin infections (viral, bacterial, or fungal), itching, and dry skin. This route is also used, in certain embodiments, to administer a composition of the present invention when a systemic, transdermal, result is desired. Such transdermal delivery is used in certain embodiments to give a steady-state level of the composition in the bloodstream over time. In certain embodiments, a permeation enhancing substance is used and the formulation may be, for example, an ointment, cream, lotion, solution, powder, gel, etc.

In certain embodiments, a composition of the present invention is administered via transdermal delivery. In certain embodiments, a composition is delivered body-wide (i.e., systemically) through a patch on the skin, for example. In certain embodiments, a composition of the present invention is mixed with a substance (such as alcohol or an oil) that enhances penetration through the skin and into the bloodstream. Many penetration enhancers are known in the art and may be used. Through a patch, a composition can be delivered slowly and continuously for many hours or days or even longer. As a result, levels of a composition in the blood can be kept relatively constant. Patches are particularly useful for compositions that are quickly eliminated from the body because the transdermal application supplies the composition continuously.

Lipid Delivery Composition

In certain embodiments, a therapeutic composition of the present is further combined with a carrier. Certain embodiments include lipid carriers, for example: a phospholipid (which can be considered a surfactant), a lipid micelle or a liposome.

Pro-Agents and Pro-Enhancers

In certain embodiments, a pro-drug (I.e., a pro-cannabinoid agent and a pro-cannabinoid enhancer) is provided and may be administered. In certain embodiments of the present invention, a pro-cannabinoid agent is administered and metabolizes to a cannabinoid agent in the subject (e.g., through a metabolic pathway). In certain embodiments of the present invention, a pro-cannabinoid enhancer is administered and metabolizes to a cannabinoid enhancer in the subject (e.g., through a metabolic pathway).

Therapeutics and Therapies

Certain embodiments of the present invention provide compositions that can be used, in an effective amount, as a therapeutic for treating conditions or diseases or for ameliorating a symptom thereof. A composition of the present invention can be used for treating a disease, disorder or condition of a mammal, including livestock, pets, service animals and, preferably, humans having such issue and in need of treatment therefor.

Memory

In certain embodiments, a therapeutic of the present invention is useful for treating memory deficits in chronic neurological conditions (for example, in Alzheimer's disease). Although it considered herein that a therapeutic of the present invention may limit the conversion of short term memory into long term memory in health individuals and in an acute sense, it is considered herein that the same therapeutic is useful to treat chronic memory deficits, preferably those memory deficits associated with a neurological disease, disorder or condition (preferably chronic conditions and the like).

Hippocampal Neurogenesis

In the adult brain, the endocannabinoid system facilitates the neurogenesis of hippocampal granule cells. In the sub-granular zone of the dentate gyrus, multipotent neural progenitors (NP) give rise to daughter cells that, over the course of several weeks, mature into granule cells whose axons project to and synapse onto dendrites on the CA3 region. NPs in the hippocampus have been shown to possess fatty acid amide hydrolase (FAAH) and express CB1 and utilize 2-AG. CB1 activation by endogenous or exogenous cannabinoids promote NP proliferation and differentiation; this activation is absent in CB1 knockouts and abolished in the presence of antagonist. In certain embodiments of the present invention, it is considered that the therapeutic (agent plus enhancer) has a synergistic effect on neurogenesis and is useful as a therapeutic for subjects in need of neurogenic treatment.

Appetite

In certain embodiments of the present invention, it is considered that the therapeutic (agent plus enhancer) has a synergistic effect on appetite control and is useful as a therapeutic for subjects in need of appetite control including weight gain treatment. In certain embodiments, a composition of the present invention is administered in a dose that treats or relieves a symptom of a first condition (or first conditions), but does not cause an exacerbation of a second condition (or second conditions), where the exacerbation is associated with a higher amount of the composition being administered. Thus, in certain embodiments, it is desirous to give a dose sufficient for a therapeutic effect without increasing a non-preferred effect (e.g., a side-effect).

Anxiety

In certain embodiments, a composition is the present invention is administered to achieve an anxiolytic effect (i.e., as an anti-anxiety treatment). In certain embodiments, the therapeutic (agent plus enhancer) has a synergistic effect as an anti-anxiety factor and is useful as a therapeutic for subjects in need of treatment for anxiety, including for post-traumatic stress disorder (PTSD) which has symptoms of heightened anxiety.

Immune Function

In certain embodiments, a composition of the present invention is useful for modulating the immune system. For example, by decreasing an immune response or by decreasing an inflammatory response.

Multiple Sclerosis

In certain embodiments, a composition of the present invention is useful for treating multiple sclerosis or a symptom of multiple sclerosis. For example, treatment includes increasing a microglial cell proliferation by administration of an effective amount in a subject in need of such treatment.

Analgesia

In certain embodiments, a composition of the present invention is useful for treating pain such as by having an analgesic or pain relief effect in a subject in need of pain relief. In certain embodiments, a composition of the present invention is useful for treating a neuropathic pain in a subject in need of treatment for neuropathic pain.

Digestive Support

In certain embodiments, a composition of the present invention is useful for digestive support in a subject in need of or seeking digestive support. In certain embodiments, a composition of the present invention is useful for treatment of a digestive illness, for example, but not limited to: Crohn's disease, colitis, inflammation, intestinal inflammation, an ulcer, gastroesophageal reflux disease (GERD), gallstones, celiac disease, ulcerative colitis, irritable bowel syndrome, hemorrhoids, or diverticulitis in a subject in need of treatment therefor or in need of amelioration of a symptom thereof.

Sleep

Intercerebroventricular administration of anandamide in rats has been shown to decrease wakefulness and increase slow-wave sleep and REM sleep. Administration of anandamide into the basal forebrain of rats has also been shown to increase levels of adenosine, which plays a role in promoting sleep and suppressing arousal. REM sleep deprivation in rats has been demonstrated to increase CB1 receptor expression in the central nervous system. Furthermore, anandamide levels possess a circadian rhythm in the rat, with levels being higher in the light phase of the day, which is when rats are usually asleep or less active, as they are nocturnally active. In certain embodiments, a composition of the present invention, improves sleep for example by increasing sleep duration, quality or both. In certain embodiments, therapy is achieved by increasing a slow-wave sleep (delta-wave sleep) or an REM sleep in a subject in need thereof. In certain embodiments, a compositon of the present invention is useful as a therapeutic for subjects in need of treatment for a sleep disorder including narcolepsy, daytime sleepiness, insomnia and restless sleep.

Nutritional Supplement

In certain embodiments, it is provided that a therapeutic of the present invention (e.g, an agent and enhancer combination) is supplied as a supplement, such as a nutritional supplement.

Treatment

In certain embodiments, a therapeutic amount of a composition of the present invention is administered for treating a disease or condition, or a symptom of a disease or condition, in a mammalian subject, preferably a human, in need of such treating or therapy to improve the disease or condition or a symptom thereof.

1. A composition for administration to a mammal, comprising: a cannabinoid agent, an enhancer and a carrier suitable for administration, wherein the enhancer increases an activity of the cannabinoid receptor agent.

2. The composition of claim 1, wherein the cannabinoid agent is an anandamide or a 2-arachidonylglycerol.

3. The composition of claim 1, wherein the enhancer inhibits an enzymatic activity of a fatty acid amide hydrolase or a monoacylglycerol lipase, thereby increasing the activity of the cannabinoid agent.

4. The composition of claim 1, wherein a dose the cannabinoid agent is an anandamide and the enhancer is a cannabidiol.

5. The composition of claim 1, wherein a dose the composition includes at least 1 mg of the agent and at least 1 mg of the enhancer.

6. The composition of claim 1, wherein a dose of the composition includes at least 5 mg of the agent and at least 1 mg of the enhancer.

7. The composition of claim 1, wherein the cannabinoid agent is a 2-arachidonylglycerol and the enhancer is a cannabidiol.

8. The composition of claim 1, wherein a dose of the composition includes at least 1 mg of the 2-arachidonylglycerol and at least 1 mg of the enhancer.

9. The composition of claim 1, wherein a dose of the composition includes at least 5 mg of the 2-arachidonylglycerol and at least 1 mg of the enhancer.

10. The composition of claim 1, further comprising a caryophyllene.

11. An article of manufacture, comprising: a measured dose device adapted for an administration of a preselected amount of a composition, wherein the composition includes a cannabinoid agent, and an enhancer, wherein the enhancer increases an activity of the cannabinoid agent.

12. The article of manufacture of claim 11, wherein the cannabinoid agent is an anandamide or a 2-arachidonylglycerol.

13. The article of manufacture of claim 11, wherein the enhancer inhibits a fatty acid amide hydrolase or a monoacylglycerol lipase, thereby inhibiting the breakdown of the cannabinoid agent and increasing the activity of the cannabinoid agent.

14. The article of manufacture of claim 11, wherein the cannabinoid agent is an anandamide and the enhancer is a cannabidiol.

15. The article of manufacture of claim 11, wherein the cannabinoid agent is a 2-arachidonylglycerol and the enhancer is a cannabidiol.

16. The article of manufacture of claim 11, wherein the composition further comprises a caryophyllene.

17. The article of manufacture of claim 11, wherein a dose of the composition includes at least 1 mg of the cannabinoid agent and 1 mg of the enhancer.

18. The article of manufacture of claim 11, wherein a dose of the composition includes at least 10 mg of the cannabinoid agent and 1 mg of the enhancer.

19. The article of manufacture of claim 11, wherein a dose of the composition includes at least 50 mg of the cannabinoid agent and 20 mg of the enhancer.

20. The article of manufacture of claim 11, wherein a dose of the composition includes at least 50 mg of the cannabinoid agent and 10 mg of the enhancer.
